(19) United States
(12) Patent Application Publication
(10) Pub. No.: US 2025/0025087 A1
(43) Pub. Date: Jan. 23, 2025

(54) METHOD AND SYSTEM FOR VISUALIZING ABLATION PROCEDURE DATA

(71) Applicant: St. Jude Medical, Cardiology Division, Inc., St. Paul, MN (US)

(72) Inventor: Nathan Matlock, Fairfield, CA (US)

(21) Appl. No.: 18/708,935

(22) PCT Filed: Nov. 10, 2022
(86) PCT No.: PCT/US2022/079671

§ 371 Date: May 9, 2024

Related U.S. Application Data
(60) Provisional application No. 63/290,986, filed on Dec. 17, 2021.

(51) Int. Cl. A61B 5/339; A61B 5/367; A61B 18/00; A61B 18/14; A61B 34/20; A61B 90/00

(52) U.S. Cl. CPC A61B 5/339; A61B 5/367; A61B 18/1492; A61B 34/20; A61B 2018/00375; A61B 2018/00577; A61B 2018/00839; A61B 2090/065

(57) ABSTRACT

Data collected by an electrophysiology catheter during an ablation procedure. such as a pulmonary vein isolation, can be visualized using an electroanatomical mapping system. The mapping system receives data points measured by the catheter, each of which includes localization information and at least one procedure metric. The system then applies an ablation procedure model to the data points. The model facilitates relating the data points to one or more anatomical features using the localization data and/or relating the data points to an ablation procedure outcome using the metric(s). This allows the system to generate map(s) of the metric(s) and output visualization(s) of the map(s). The visualization(s) can include two-dimensional schematic anatomical representation(s) of the metric(s) and/or two-dimensional representation(s) of the metric(s) versus time. Various metrics, including, but not limited to, contact angle, contact force, and catheter stability, can be visualized.

### CROSS-REFERENCE TO RELATED APPLICATIONS

This application claims the benefit of U.S. provisional application No. 63/290,986, filed 17 Dec. 2021, which is hereby incorporated by reference as though fully set forth herein.

### BACKGROUND

The present disclosure relates generally to electrophysiology procedures, such as cardiac diagnostic and therapeutic procedures, including electrophysiological mapping and cardiac ablation. In particular, the present disclosure relates to visualizing data collected during a cardiac ablation procedure, such as a pulmonary vein isolation procedure.

Electrophysiological mapping, and more particularly electrocardiographic mapping, is a part of numerous cardiac diagnostic and therapeutic procedures. The electroanatomical mapping systems used during such procedures collect a large amount of data, and often display that data to a practitioner in real time. Although these data are stored, for example to review a particular electrophysiology procedure, they have additional value in other contexts. In particular, it would be advantageous, however, to leverage these data to inform future electrophysiology procedures.

### BRIEF SUMMARY

The instant disclosure provides a method of visualizing data collected by an electrophysiology catheter during an ablation procedure. The method includes: receiving a plurality of data points measured by the electrophysiology catheter at an electroanatomical mapping system, each data point of the plurality of data points including localization information and at least one procedure metric; applying an ablation procedure model to the plurality of data points via the electroanatomical mapping system, thereby generating a map of the at least one procedure metric; and outputting a visualization of the map of the at least one procedure metric via the electroanatomical mapping system. In embodiments of the disclosure, the ablation procedure is a pulmonary vein isolation procedure.

The visualization can include a two-dimensional schematic anatomical representation of the at least one procedure metric. Alternatively or additionally, the visualization and include a two-dimensional representation of the at least one procedure metric versus time.

It is contemplated that the at least one procedure metric can be an angle of contact between the electrophysiology catheter and a tissue being ablated, stability of the electrophysiology catheter relative to a tissue being ablated, and/or contact force between the electrophysiology catheter and a tissue being ablated.

The method can also include adding the plurality of data points measured by the electrophysiology catheter to the ablation procedure model.

Advantageously, the ablation procedure model can facilitate relating the plurality of data points to one or more anatomical features using the localization data of the plurality of data points. It can also facilitate relating the plurality of data points to an ablation procedure outcome using the at least one procedure metric of the plurality of data points.

Also disclosed herein is a method of visualizing a pulmonary vein isolation procedure, including the steps of: receiving a plurality of data points measured by an electrophysiology catheter during the pulmonary vein isolation procedure at an electroanatomical mapping system, each data point of the plurality of data points including localization information and at least one procedure metric; applying an ablation procedure model to the plurality of data points via the electroanatomical mapping system, thereby generating a map of the at least one procedure metric; and outputting a two-dimensional visualization of the map of the at least one procedure metric via the electroanatomical mapping system.

The at least one procedure metric can be an angle of contact between the electrophysiology catheter and a pulmonary vein being ablated, stability of the electrophysiology catheter relative to a pulmonary vein being ablated, and/or contact force between the electrophysiology catheter and a pulmonary vein being ablated.

It is contemplated that the plurality of data points measured by the electrophysiology catheter can be added to the ablation procedure model.

Advantageously, the ablation procedure model facilitates relating the plurality of data points to one or more pulmonary veins using the localization data of the plurality of data points. It can also advantageously facilitate relating the plurality of data points to an outcome of the pulmonary vein isolation procedure using the at least one procedure metric of the plurality of data points.

The instant disclosure also provides an electroanatomical mapping system including an ablation procedure visualization module. This module is configured to: receive a plurality of data points measured by an electrophysiology catheter, each data point of the plurality of data points including localization information and at least one procedure metric; apply an ablation procedure model to the plurality of data points, thereby generating a map of the at least one procedure metric; and output a visualization of the map of the at least one procedure metric. The visualization can include a two-dimensional schematic anatomical representation of the at least one procedure metric and/or a two-dimensional representation of the at least one procedure metric versus time.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of an exemplary electroanatomical mapping system.

FIG. 2 depicts an exemplary catheter that can be used in connection with aspects of the instant disclosure.

FIG. 3 is a flowchart of representative steps that can be carried out according to aspects of the instant disclosure.

FIG. 4 is a first representative visualization of a procedure metric according to aspects of the instant disclosure.

FIG. 5 is a second representative visualization of a procedure metric according to additional aspects of the instant disclosure.

FIG. 6 is a third representative visualization of a procedure metric according to aspects of the instant disclosure.

FIG. 7 is a fourth representative visualization of a procedure metric according to aspects of the instant disclosure.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure provides systems, apparatuses, and methods for visualizing data collected during an electrophysiology procedure. For purposes of illustration, aspects of the disclosure will be described with reference to visualizing data collected during a pulmonary vein isolation (PVI) procedure. As those of ordinary skill in the art will readily appreciate, PVI procedures can be carried out, by way of example, using a radiofrequency (RF) ablation catheter such as the TactiCath™ Contact Force Ablation Catheter, Sensor Enabled™, from Abbott Laboratories (Abbott Park, Illinois), which may be used in conjunction with an electroanatomical mapping system, such as the EnSite Precision™ cardiac mapping system or the Ensite™ X EP System, both also from Abbott Laboratories. Those of ordinary skill in the art will understand, however, how to apply the teachings herein to good advantage in other contexts and/or with respect to other devices.

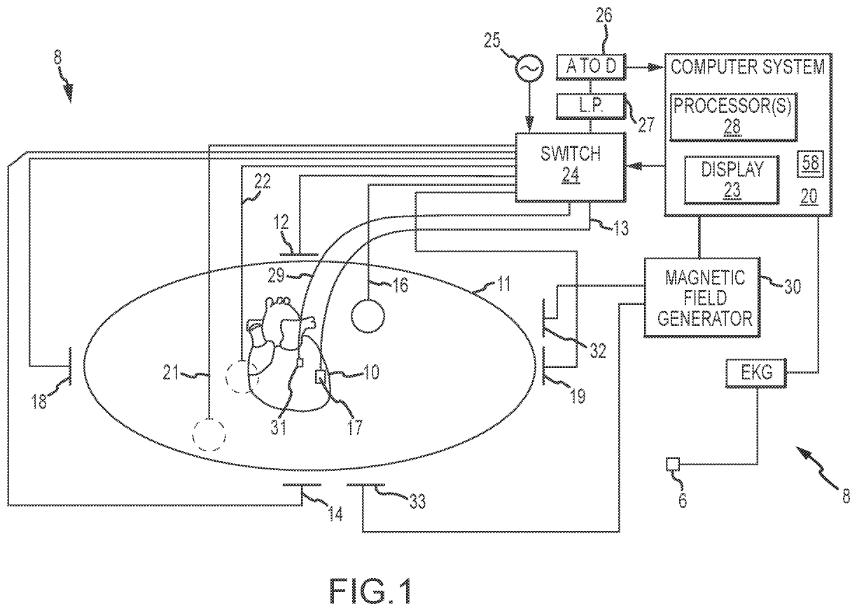

FIG. 1 shows a schematic diagram of an exemplary electroanatomical mapping system 8 for conducting cardiac electrophysiology procedures by navigating a cardiac catheter through a patient's vasculature to a desired site (e.g., within heart 10 of patient 11) for diagnosis (e.g., measuring electrical activity occurring in heart 10), mapping (e.g., three-dimensionally mapping the electrical activity and/or information related to or representative of the electrical activity so measured), and/or therapy delivery (e.g., performing a PVI procedure). System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example to create a diagnostic data map of the patient's heart 10.

As one of ordinary skill in the art will recognize, system 8 determines the location, and in some aspects the orientation, of objects, typically within a three-dimensional space, and expresses those locations as position information determined relative to at least one reference. This is referred to herein as “localization.”

For simplicity of illustration, the patient 11 is depicted schematically as an oval. In the embodiment shown in FIG. 1, three sets of surface electrodes (e.g., patch electrodes) 12, 14, 16, 18, 19, and 22 are shown applied to a surface of the patient 11, pairwise defining three generally orthogonal axes, referred to herein as an x-axis (12, 14), a y-axis (18, 19), and a z-axis (16, 22). In other embodiments the electrodes could be positioned in other arrangements, for example multiple electrodes on a particular body surface. As a further alternative, the electrodes do not need to be on the body surface, but could be positioned internally to the body.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 18, 19 are applied to the patient along a second axis generally orthogonal to the x-axis, such as along the inner thigh and neck regions of the patient, and may be referred to as the Left Leg and Neck electrodes. The z-axis electrodes 16, 22 are applied along a third axis generally orthogonal to both the x-axis and the y-axis, such as along the sternum and spine of the patient in the thorax region, and may be referred to as the Chest and Back electrodes. The heart 10 lies between these pairs of surface electrodes 12/14, 18/19, and 16/22.

According to embodiments of the instant disclosure, each surface electrode measures six signals—three resistance (impedance) signals and three reactance signals. These signals can, in turn, be grouped into three resistance/reactance signal pairs. One resistance/reactance signal pair reflects driven values, as described below, while the other two resistance/reactance signal pairs reflect non-driven values (e.g., measurements of the electric field generated by other driven pairs in a manner similar to that described below for electrodes 17).

An additional surface reference electrode (e.g., a “belly patch”) 21 provides a reference and/or ground electrode for the system 8. The belly patch electrode 21 may be an alternative to a fixed intra-cardiac electrode 31, described in further detail below. In alternative embodiments, the surface reference electrode 21 can alternatively or additionally include a magnetic patient reference sensor-anterior (“PRS-A”) positioned on the patient's chest.

It should also be appreciated that, in addition, the patient 11 may have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to the system 8 (e.g., it can be provided as input to computer system 20). Insofar as ECG leads are well understood, and for the sake of clarity in the figures, only a single lead 6 and its connection to computer 20 is illustrated in FIG. 1.

A representative catheter 13 having at least one electrode 17 is also shown. This representative catheter electrode 17 is referred to as the “roving electrode,” “moving electrode,” or “measurement electrode” throughout the specification. Typically, multiple electrodes 17 on catheter 13, or on multiple such catheters, will be used. In one embodiment, for example, the system 8 may comprise sixty-four electrodes on twelve catheters disposed within the heart and/or vasculature of the patient. In other embodiments, system 8 may utilize a single catheter that includes multiple (e.g., eight) splines, each of which in turn includes multiple (e.g., eight) electrodes. The foregoing embodiments are merely exemplary, however, and any number of electrodes and/or catheters may be used.

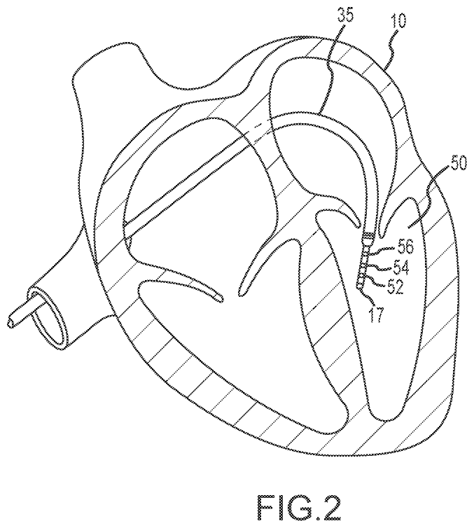

The person of ordinary skill in the art will understand that catheter 13 (or multiple such catheters) can be introduced into the vasculature and/or heart 10 via one or more introducers and using familiar procedures. For purposes of this disclosure, a segment of an exemplary catheter 13, such as the TactiCath™ Contact Force Ablation Catheter, Sensor Enabled™, is shown in FIG. 2. In FIG. 2, catheter 13 extends into the left ventricle 50 of the patient's heart 10 through a transseptal sheath 35. The use of a transseptal approach is well-known and will be familiar to those of ordinary skill in the art, and thus, need not be further described herein. Of course, catheter 13 can also be introduced into heart 10 in any other suitable manner.

Catheter 13 includes electrode 17 on its distal tip; electrode 17 may be an RF ablation electrode. Catheter 13 can also include a plurality of additional sensors 52, 54, 56 spaced along its length as illustrated. In embodiments of the disclosure, sensors 52, 54, and 56 may be additional electrodes, but it is also contemplated that sensors 52, 54, and 56 can incorporate other types of sensors including, without limitation, contact force sensors, temperature sensors, and the like.

Where sensors 52, 54, and 56 are electrodes, it is typical that the spacing therebetween will be known, though it should be understood that the electrodes may not be evenly spaced along catheter 13 or of equal size to each other. Since each of these electrodes 17, 52, 54, 56 lies within the patient, location data may be collected simultaneously for each of the electrodes by system 8.

Similarly, each of electrodes 17, 52, 54, and 56 can be used to gather electrophysiological data from the cardiac surface. The ordinarily skilled artisan will be familiar with various modalities for the acquisition and processing of electrophysiology data points (including, for example, both contact and non-contact electrophysiological mapping and the collection of both unipolar and bipolar electrograms), such that further discussion thereof is not necessary to the understanding of the techniques disclosed herein. Likewise, various techniques familiar in the art can be used to generate a graphical representation from the plurality of electrophysiology data points. Insofar as the ordinarily skilled artisan will appreciate how to create electrophysiology maps from electrophysiology data points, the aspects thereof will only be described herein to the extent necessary to understand the instant disclosure.

Returning now to FIG. 1, in some embodiments, an optional fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein.

Generally, three nominally orthogonal electric fields are generated by a series of driven and sensed electric dipoles (e.g., by driving pairs of patch electrodes 12/14, 18/19, and 16/22) in order to realize catheter navigation in a biological conductor. Alternatively, these orthogonal fields can be decomposed and any pairs of patch electrodes can be driven as dipoles to provide effective electrode triangulation. Likewise, the electrodes 12, 14, 18, 19, 16, and 22 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two patch electrodes 12, 14, 16, 18, 19, 22 may be selected as a dipole source and drain with respect to a ground reference, such as belly patch 21, while the unexcited electrodes measure voltage with respect to the ground reference. The roving electrodes 17 placed in the heart 10 are likewise exposed to the field from a current pulse and can likewise be measured with respect to ground, such as belly patch 21. In practice the catheters within the heart 10 may contain more or fewer electrodes than the sixteen shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which system 8 measures positions. Data sets from each of the surface electrodes, the internal electrodes, and the virtual electrodes may all be used to determine the location of the roving electrodes 17 within heart 10.

The measured voltages may be used by system 8 to determine the location in three-dimensional space of the electrodes inside the heart, such as roving electrodes 17 relative to a reference location, such as reference electrode 31. That is, the voltages measured at reference electrode 31 may be used to define the origin of a coordinate system, while the voltages measured at roving electrodes 17 may be used to express the location of roving electrodes 17 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, system 8 first selects a set of surface electrodes and then drives them with current pulses. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In aspects of the disclosure, system 8 can be a hybrid system that incorporates both impedance-based (e.g., as described above) and magnetic-based localization capabilities. Thus, for example, system 8 can also include a magnetic source 30, which is coupled to one or more magnetic field generators. In the interest of clarity, only two magnetic field generators 32 and 33 are depicted in FIG. 1, but it should be understood that additional magnetic field generators (e.g., a total of six magnetic field generators, defining three generally orthogonal axes analogous to those defined by patch electrode sets 12, 14, 16, 18, 19, and 22) can be used without departing from the scope of the present teachings. Likewise, those of ordinary skill in the art will appreciate that, for purposes of localizing catheter 13 within the magnetic fields so generated, catheter 13 can include one or more magnetic localization sensors (e.g., coils).

In some embodiments, system 8 is the EnSite™ Velocity™, EnSite Precision™, or EnSite™ X cardiac mapping and visualization system of Abbott Laboratories. Other localization systems, however, may be used in connection with the present teachings, including for example the RHYTHMIA HDX™ mapping system of Boston Scientific Corporation (Marlborough, Massachusetts), the CARTO navigation and location system of Biosense Webster, Inc. (Irvine, California), the AURORA® system of Northern Digital Inc. (Waterloo, Ontario), Stereotaxis, Inc.'s NIOBE® Magnetic Navigation System (St. Louis, Missouri), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

Aspects of the disclosure relate to visualizing data collected by catheter 13 during an ablation procedure, such as a PVI procedure. System 8 can therefore include an ablation procedure visualization module 58.

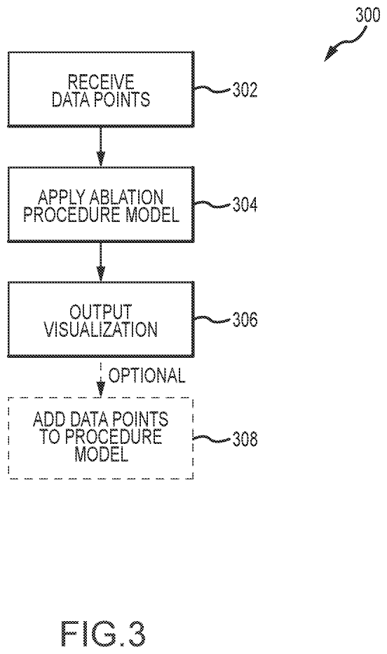

One exemplary method according to aspects of the instant disclosure will be explained with reference to the flowchart 300 of representative steps presented as FIG. 3. In some embodiments, for example, flowchart 300 may represent several exemplary steps that can be carried out by electroanatomical mapping system 8 of FIG. 1 (e.g., by processor 28 and/or ablation procedure visualization module 58). It should be understood that the representative steps described below can be either hardware-or software-implemented. For the sake of explanation, the term “signal processor” may be used to describe both hardware-and software-based implementations of the teachings herein.

In block 302, system 8 receives a plurality of data points measured by catheter 13. Each of the received data points includes both localization information (that is, information regarding the position and/or orientation of catheter 13 when the data point was collected) and at least one procedure metric.

The term “procedure metric” is used herein to describe data, other than localization information, collected by electrodes and/or sensors (e.g., 17, 52, 54, 56) on catheter 13. Insofar as extant electroanatomical mapping systems are known to collect various procedure metrics, and to display them to practitioners through their respective graphical user interfaces (e.g., as numerical values), the ordinarily-skilled artisan will be familiar with a variety of procedure metrics that may be presented to good advantage in connection with the teachings herein. By way of example only, however, procedure metrics that may be desirable within the context of a PVI procedure include, without limitation, an angle of contact between catheter 13 and the tissue being ablated; measures of stability of catheter 13 vis-à-vis the tissue being ablated; the contact force between catheter 13 and the tissue being ablated; the temperature of the tissue being ablated; the impedance of the tissue being ablated; properties of the current being delivered to the tissue being ablated (e.g., duration, amplitude, phase angle, and the like); and the like. Procedure metrics may also include composite metrics derived from multiple individual metrics (e.g., changes in the temperature of the tissue being ablated over time, or the time derivative of the impedance of the tissue being ablated).

In block 304, electroanatomical mapping system 8 applies a model to the data points received in block 302. The model applied in block 304, referred to herein as an “ablation procedure model,” is developed using previously-collected data (e.g., localization information and procedure metrics). The data underlying the procedure model may be limited to data gathered during similar procedures (e.g., other PVI procedures), conducted using the same electroanatomical mapping system 8 and/or by the same practitioner; alternatively, the data underlying the procedure model may be more general in nature, including data from additional procedures, additional practitioners, and/or additional systems.

In any event, these data enable the procedure model to facilitate relating the plurality of data points received in block 302 to anatomical features (e.g., pulmonary veins) using the localization data of the plurality of data points. Similarly, the ablation procedure model can facilitate relating the plurality of data points to an ablation procedure outcome (e.g., successful isolation of a pulmonary vein) using the at least one procedure metric of the plurality of data points.

The output of the application of the ablation procedure model to the data points received in block 302 is referred to herein as a “map” of the at least one procedure metric. In block 306, electroanatomical mapping system 8 outputs a visualization of this map, e.g., on display 23.

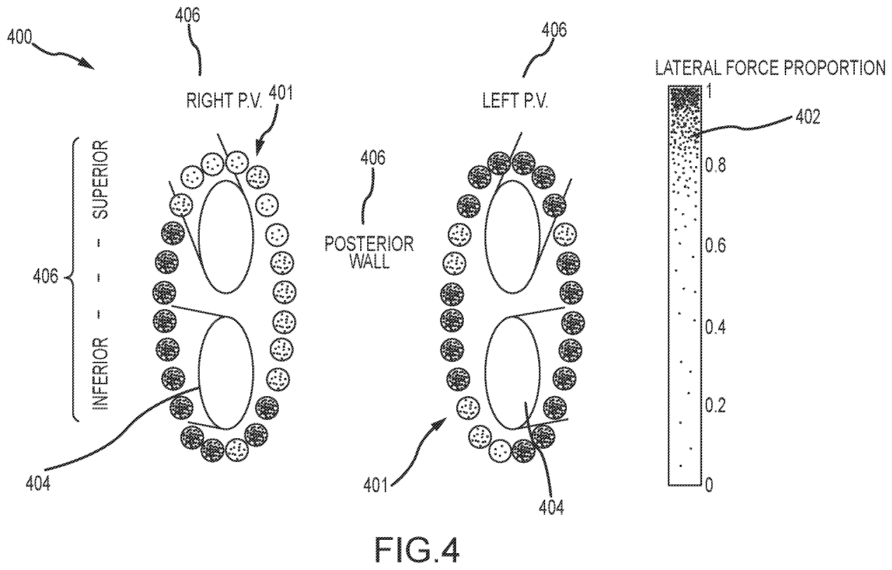

FIG. 4 depicts a visualization of a map of a procedure metric according to certain embodiments of the instant disclosure. More particularly, FIG. 4 depicts a two-dimensional schematic anatomical representation 400 of the procedure metric which, in the embodiment illustrated, is the lateral component of the contact force between catheter 13 and the tissue being ablated. Values of the procedure metric (that is, contact force) are shown as dots 401 (or other glyphs) and are presented in greyscale 402 or a similar display convention, such as a color- or pattern-scale.

To provide a practitioner with anatomical references that aid in the interpretation of schematic anatomical representation 400, the visualization includes a schematic representation of pulmonary veins 404, as well as labels 406 indicating right, left, posterior, inferior, and superior directions.

Each dot 401 in representation 400 reflects the value of the procedure metric for one or more of the data points received in block 302. That is, each dot 401 in representation 400 can correspond to a single data point or to a cluster of data points that are relatively close to each other, as determined by their respective localization information (e.g., clustering data points that fall within a preset Euclidean distance of a particular seed data point). Various approaches are contemplated to determine the displayed value of the procedure metric for a dot 401 that represents a cluster of data points, including the mean value of the procedure metric across all data points in the cluster, the median value of the procedure metric across all data points in the cluster, or the mode of the procedure metric across all data points in the cluster.

Each dot 401 is likewise associated with localization information. Where dot 401 corresponds to a single data point, the localization information of dot 401 can be identical to the localization information of the corresponding data point. Where dot 401 represents a cluster of data points, on the other hand, its localization information can be derived from the localization information for the data points in the cluster-for example, the localization information of dot 401 can be defined as the centroid of the cluster.

As described above, the ablation procedure model facilitates relating the plurality of data points received in block 302 to anatomical features (e.g., pulmonary veins), and thus informs where on representation 400 each dot 401 appears. Stated differently, given the localization information for a dot 401, the ablation procedure model determines the left/right, anterior/posterior, and inferior/superior positioning of that dot within representation 400. Advantageously, therefore, the ablation procedure model allows representation 400 to be created, and for the data points received in block 302 to be represented thereon, without requiring the creation of a full, or even a partial, cardiac geometry.

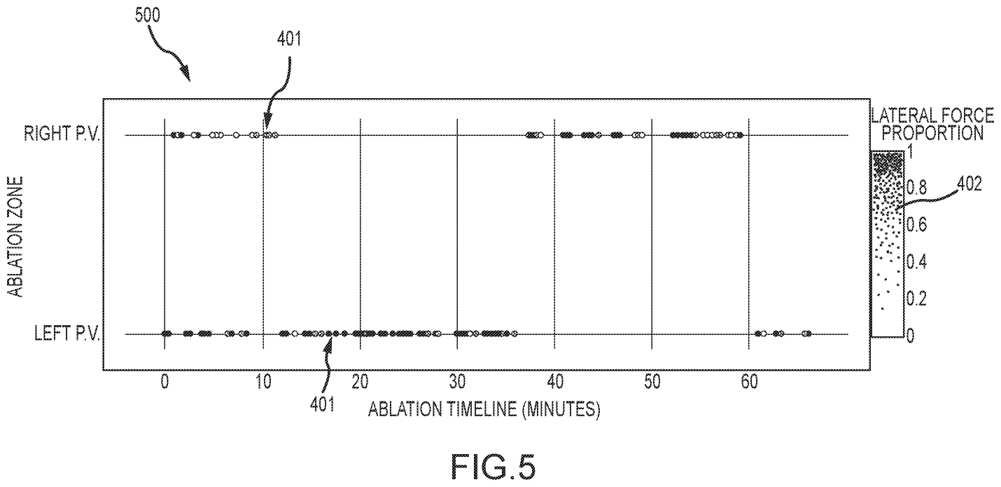

FIG. 5 depicts an additional visualization of a map of a procedure metric according to additional embodiments of the instant disclosure. More particularly, FIG. 5 depicts a two-dimensional representation 500 of the procedure metric (which, for purposes of illustration, is once again the lateral component of the contact force between catheter 13 and the tissue being ablated) versus time. Once again, the procedure model allows system 8 to determine whether any given dot 401 should be represented at the right or left pulmonary vein (as shown on the y-axis), while the position of dot 401 along the x-axis is determined by the time of its collection. Similar to determining the localization information and/or procedure metric value for a dot 401 that corresponds to a cluster of data points, the time stamp for a dot 401 can also be determined from the time of collection of its constituent data points (e.g., the mean collection time across its constituent data points).

Returning now to FIG. 3, in optional block 308, the data points received in block 302 can be added to the procedure model.

Although several embodiments have been described above with a certain degree of particularity, those skilled in the art could make numerous alterations to the disclosed embodiments without departing from the spirit or scope of this invention.

For example, the teachings herein can be applied in real time (e.g., during an ablation procedure) or during post-processing (e.g., to data points collected during an ablation procedure or electrophysiology study performed at an earlier time).

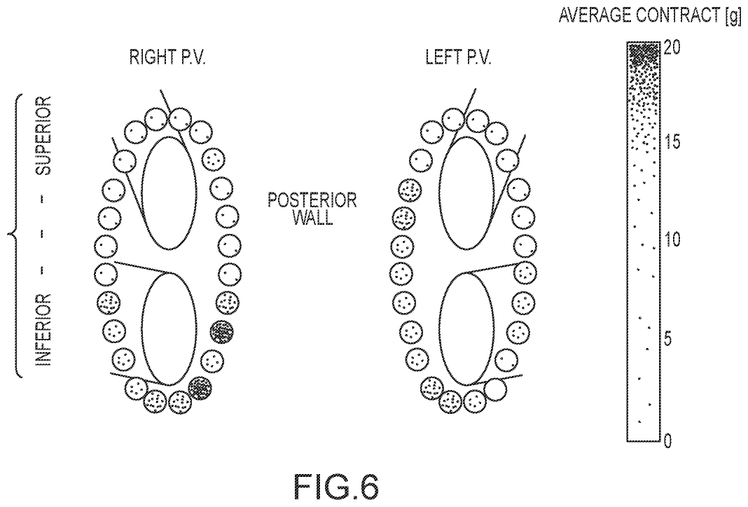

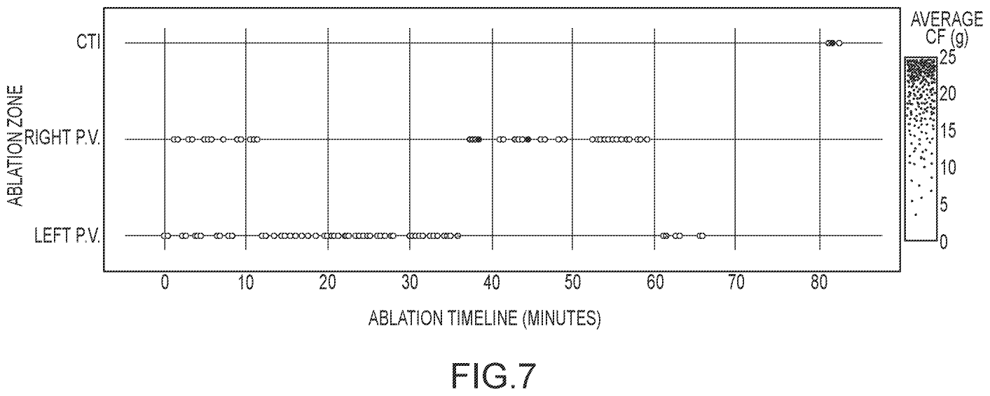

As additional examples, FIGS. 6 and 7 depict visualizations, analogous to FIGS. 4 and 5, respectively, of total contact force between catheter 13 and the tissue being ablated (with each dot representing an average total contact force for a cluster of data points).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of visualizing data collected by an electrophysiology catheter during an ablation procedure, the method comprising:
receiving a plurality of data points measured by the electrophysiology catheter at an electroanatomical mapping system, each data point of the plurality of data points including localization information and at least one procedure metric;
applying an ablation procedure model to the plurality of data points via the electroanatomical mapping system, thereby generating a map of the at least one procedure metric; and
outputting a visualization of the map of the at least one procedure metric via the electroanatomical mapping system.

2. The method according to claim 1, wherein the ablation procedure comprises a pulmonary vein isolation procedure.

3. The method according to claim 2, wherein the visualization comprises a two-dimensional schematic anatomical representation of the at least one procedure metric.

4. The method according to claim 2, wherein the visualization comprises a two-dimensional representation of the at least one procedure metric versus time.

5. The method according to claim 1, wherein the at least one procedure metric comprises an angle of contact between the electrophysiology catheter and a tissue being ablated.

6. The method according to claim 1, wherein the at least one procedure metric comprises stability of the electrophysiology catheter relative to a tissue being ablated.

7. The method according to claim 1, wherein the at least one procedure metric comprises contact force between the electrophysiology catheter and a tissue being ablated.

8. The method according to claim 1, further comprising adding the plurality of data points measured by the electrophysiology catheter to the ablation procedure model.

9. The method according to claim 1, wherein the ablation procedure model facilitates relating the plurality of data points to one or more anatomical features using the localization data of the plurality of data points.

10. The method according to claim 1, wherein the ablation procedure model facilitates relating the plurality of data points to an ablation procedure outcome using the at least one procedure metric of the plurality of data points.

11. A method of visualizing a pulmonary vein isolation procedure, the method comprising:
receiving a plurality of data points measured by an electrophysiology catheter during the pulmonary vein isolation procedure at an electroanatomical mapping system, each data point of the plurality of data points including localization information and at least one procedure metric;
applying an ablation procedure model to the plurality of data points via the electroanatomical mapping system, thereby generating a map of the at least one procedure metric; and
outputting a two-dimensional visualization of the map of the at least one procedure metric via the electroanatomical mapping system.

12. The method according to claim 11, wherein the at least one procedure metric comprises an angle of contact between the electrophysiology catheter and a pulmonary vein being ablated.

13. The method according to claim 11, wherein the at least one procedure metric comprises stability of the electrophysiology catheter relative to a pulmonary vein being ablated.

14. The method according to claim 11, wherein the at least one procedure metric comprises contact force between the electrophysiology catheter and a pulmonary vein being ablated.

15. The method according to claim 11, further comprising adding the plurality of data points measured by the electrophysiology catheter to the ablation procedure model.

16. The method according to claim 11, wherein the ablation procedure model facilitates relating the plurality of data points to one or more pulmonary veins using the localization data of the plurality of data points.

17. The method according to claim 11, wherein the ablation procedure model facilitates relating the plurality of data points to an outcome of the pulmonary vein isolation procedure using the at least one procedure metric of the plurality of data points.

18. An electroanatomical mapping system, comprising:
an ablation procedure visualization module configured to:
receive a plurality of data points measured by an electrophysiology catheter, each data point of the plurality of data points including localization information and at least one procedure metric;
apply an ablation procedure model to the plurality of data points, thereby generating a map of the at least one procedure metric; and
output a visualization of the map of the at least one procedure metric.

19. The system according to claim 18, wherein the visualization of the map of the at least one procedure metric comprises a two-dimensional schematic anatomical representation of the at least one procedure metric.

20. The system according to claim 18, wherein the visualization of the map of the at least one procedure metric comprises a two-dimensional representation of the at least one procedure metric versus time.
